(19) United States
(12) Patent Application Publication
(10) Pub. No.: US 2009/0182153 A1
(43) Pub. Date: Jul. 16, 2009

(54) PROCESS FOR PRODUCTION OF OPTICALLY ACTIVE ALLYL COMPOUND

(75) Inventor: Yoshikazu Ootsuka, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Nissan Chemical Industries, LTD, Chiyoda-ku (JP)

(21) Appl. No.: 12/278,582

(22) PCT Filed: Feb. 8, 2006
(86) PCT No.: PCT/JP2007/052284

§ 371 Date: Aug. 7, 2008

(30) Foreign Application Priority Data
Feb. 8, 2006 (JP) 2006-030964

(51) Int. Cl. C07D 209/32; C07C 67/00; C07C 45/00

(52) U.S. Cl. 548/485; 560/122; 568/356

(57) ABSTRACT

To provide a novel process for producing an optically active allyl compound which is useful as an intermediate raw material for e.g. pharmaceutical products.

A process for producing an optically active allyl compound of the formula (4):

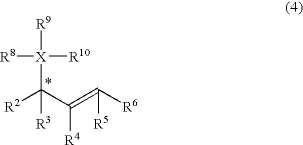

(wherein “*” represents an asymmetric carbon atom), which comprises reacting an allyloxy compound of the formula (1):

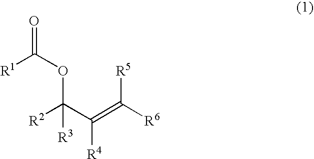

(wherein R¹ is a C₁₋₆ alkyl group or a C₁₋₆ alkoxyl group, and each of R², R³, R⁴, R⁵ and R⁶ independently is a C₁₋₆ alkyl group which may be linear, branched or cyclic, a hydrogen atom or a C₆₋₁₂ aromatic group, provided that R² and R⁶ may be located in the same ring) with a hydrogenated compound of the formula (3):

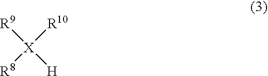

(wherein X is a carbon atom, an oxygen atom, a sulfur atom or a nitrogen atom, and each of R⁸, R⁹ and R¹⁰ independently is a C₁₋₂₄ alkyl group which may be linear, branched or cyclic, a C₁₋₂₄ alkylcarbonyl group which may be branched or cyclic, a C₁₋₂₄ alkoxycarbonyl group which may be branched or cyclic, a hydrogen atom, a halogen atom or a C₆₋₁₀ aromatic group, or two of R⁸, R⁹ and R¹⁰ may together form a ring containing one or two carbonyl groups), in the presence of a palladium compound and an optically active phosphine ligand of the formula (2):

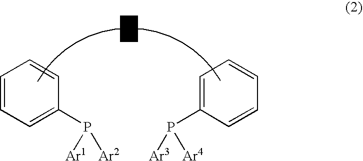

(wherein each of Ar¹, Ar², Ar³ and Ar⁴ independently is a C₆₋₁₀ aromatic group, and R⁷ is a structure having at least one asymmetric center or axial chirality), wherein a tertiary amine of the formula (5):

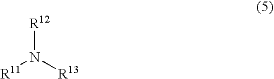

(wherein each of R¹¹, R¹² and R¹³ independently is a C₂₋₁₂ aliphatic group or a C₂₋₁₂ substituted aliphatic group, which may be linear, branched or cyclic, or a C₆₋₁₀ aromatic group or a C₆₋₁₀ substituted aromatic group) is present in the above reaction system.

### TECHNICAL FIELD

In a process for producing an optically active allyl compound, the present invention provides a novel process which is excellent in operation efficiency, can be operated at low cost, and has high optical selectivity.

### BACKGROUND ART

A process for producing an optically active allyl compound has been desired, and specifically, an asymmetric synthetic reaction using a catalyst made of a combination of a palladium compound and an optically active phosphine ligand, has been actively studied (e.g. Non-patent Document 1). With respect to using a base as a reaction reagent, (1) a method of using sodium hydride, (2) a method of using N,O-bis(trimethylsillyl)acetamide (e.g. Non-patent Document 1), (3) a method of using cesium carbonate (e.g. Non-patent Document 2) and (4) a method of using sodium hydride and a halogenated quaternary ammonium salt (e.g. Non-patent Document 3), are known.

Non-patent Document 1: Chemical Review, Vol. 103, p. 2921 (2003)

Non-patent Document 2: Angewandte Chemie International Edition in English, Vol. 35, p. 100 (1996)

Non-patent Document 3: Journal of the American Chemical Society, Vol. 116, p. 4089 (1994)

### DISCLOSURE OF THE INVENTION

### Problems to be Solved by the Invention

However, the above methods had the following problems respectively, whereby it was difficult to practice a mass production.

(1) With respect to the method of using sodium hydride, the reaction system tends to be gelled, so that stirring will become impossible. Further, the optical purity of an optically active allyl compound as the product, is low.

(2) With respect to the method of using N,O-bis(trimethylsillyl)acetamide, the reagent is expensive. Further, the optical purity of an optically active allyl compound as the product, is low.

(3) With respect to the method of using cesium carbonate, the reagent is expensive. Further, the reaction solution becomes a slurry, and a solid precipitates, whereby it is difficult to withdraw it during a mass production.

(4) With respect to the method of using sodium hydride and a halogenated quaternary ammonium salt, the reaction system tends to be gelled, so that stirring will become impossible. Further, the reagent is expensive. Furthermore, reproductivity of the reaction is low, and the optical purity of an optically active allyl compound as the product, fluctuates.

Therefore, it has been desired to develop a novel process which is excellent in operation efficiency, can be operated at low cost, and has high optical selectivity, which are difficult to attain by the conventional methods.

### Means to Solve the Problems

The present inventors have conducted extensive studies to overcome the above problems, and as a result, they have found that by using a tertiary amine, the above problems can be solved. The present invention has been accomplished on the basis of the discovery.

Namely, the present invention provides the following.

(1) A process for producing an optically active allyl compound of the formula (4):

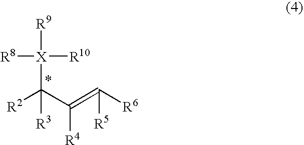

(wherein “*” represents an asymmetric carbon atom), which comprises reacting an allyloxy compound of the formula (1):

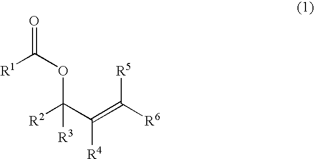

(wherein R¹ is a C₁₋₆ alkyl group or a C₁₋₆ alkoxyl group, and each of R², R³, R⁴, R⁵ and R⁶ independently is a C₁₋₆ alkyl group which may be linear, branched or cyclic, a hydrogen atom or a C₆₋₁₂ aromatic group, provided that R² and R⁶ may be located in the same ring) with a hydrogenated compound of the formula (3):

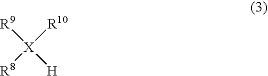

(wherein X is a carbon atom, an oxygen atom, a sulfur atom or a nitrogen atom, and each of R⁸, R⁹ and R¹⁰ independently is a C₁₋₂₄ alkyl group which may be linear, branched or cyclic, a C₁₋₂₄ alkylcarbonyl group which may be branched or cyclic, a C₁₋₂₄ alkoxycarbonyl group which may be branched or cyclic, a hydrogen atom, a halogen is atom or a C₆₋₁₀ aromatic group, or two of R⁸, R⁹ and R¹⁰ may together form a ring containing one or two carbonyl groups), in the presence of a palladium compound and an optically active phosphine ligand of the formula (2):

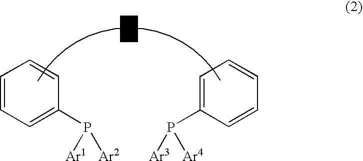

(wherein each of Ar¹, Ar², Ar³ and Ar⁴ independently is a C₆₋₁₀ aromatic group, and R⁷ is a structure having at least one asymmetric center or axial chirality), wherein a tertiary amine of the formula (5):

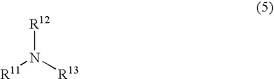

(wherein each of R¹¹, R¹² and R¹³ independently is a C₂₋₁₂ aliphatic group or a C₂₋₁₂ substituted aliphatic group, which may be linear, branched or cyclic, or a C₆₋₁₀ aromatic group or a C₆₋₁₀ substituted aromatic group) is present in the above reaction system.
(2) The process according to the above (1), wherein the optically active phosphine ligand is a compound of the formula (6):

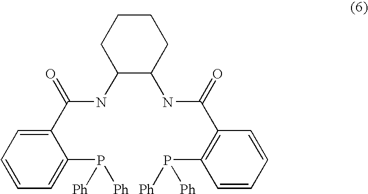

(3) The process according to the above (1), wherein the optically active phosphine ligand is a compound of the formula (7):

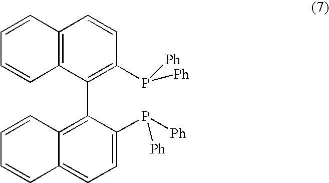

(4) The process according to the above (1), wherein the allyloxy compound is cyclopentenyl acetate of the formula (8):

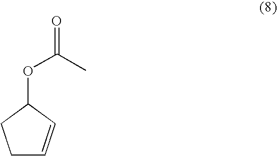

(5) The process according to the above (1), wherein the allyloxy compound is a compound of the formula (9):

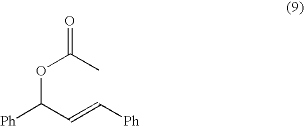

(6) The process according to the above (1), wherein the tertiary amine is tri-n-propylamine.
(7) The process according to the above (1), wherein the tertiary amine is tri-n-octylamine.
(8) The process according to the above (1), wherein the tertiary amine is diisopropylethylamine.
(9) The process according to the above (4), wherein the hydrogenated compound is a compound of the formula (10):

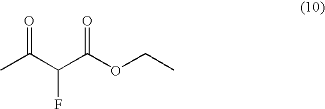

(10) The process according to the above (1), wherein the hydrogenated compound has a pKa of at most 16 in water.

### BEST MODE FOR CARRYING OUT THE INVENTION

Now, the present invention will be described in further detail. In the definitions of the compounds in the present specification, for example, “C₁₋₆” means having from 1 to 6 carbon atoms, and “C₁₋₂₄”, “C₂₋₁₂”, “C₆₋₁₂”, “C₆₋₁₀”, etc. have the corresponding meanings, respectively.

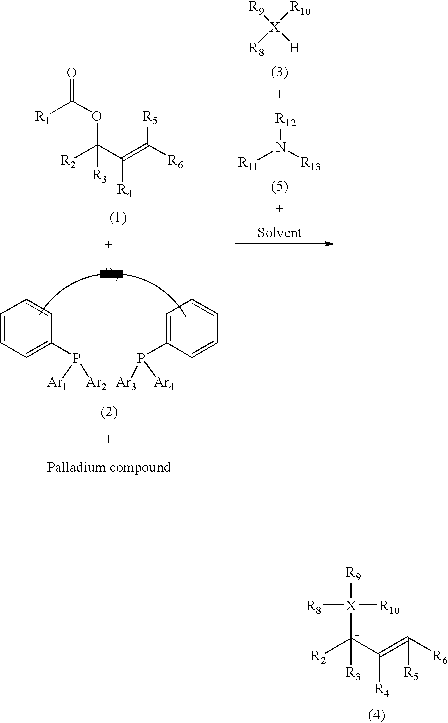

As shown in the above reaction scheme, in the present invention, in a solvent, an optically active phosphine ligand of the formula (2) and a palladium compound are added to an allyloxy compound of the formula (1), and a hydrogenated compound of the formula (3) and a tertiary amine of the formula (5) are further added thereto, whereby it is possible to produce an optically active allyl compound of the formula (4).

As the allyloxy compound of the formula (1), it is possible to use either an optically active form or a racemic modification. It may, for example, be cyclopentenyl acetate, diphenylallyl acetate or cyclopentenylmethyl carbonate.

The optically active phosphine ligand of the formula (2) may, for example, be 1,2-diaminocyclohexane-N,N′-bis(2′-diphenylphosphinobenzoyl), 1,2-diaminocyclohexane-N,N′-bis(2′-diphenylphosphinonaphthoyl), 1,2-diaminodiphenylethane-N,N′-bis(2′-diphenylphosphinobenzoyl), 2,2′-bis(diphenylphosphino)-1,1′-binaphthyl or 2,2′-bis(di(3,5-xylyl)phosphino)-1,1′-binaphthyl.

Further, the absolute configuration of the product is determined by the absolute steric configuration of the optically active phosphine ligand. For example, when the optically active phosphine ligand is (S,S)-1,2-diaminocyclohexane-N,N′-bis(2′-diphenylphosphino benzoyl), (S,S)-1,2-diaminocyclohexane-N,N′-bis(2′-diphenylphosphinonaphthoyl), (S,S)-1,2-diaminodiphenyl ethane-N,N′-bis(2′-diphenylphosphinobenzoyl), (R)-2,2′-bis(diphenylphosphino)-1,1′-binaphthyl or (R)-2,2′-bis(di(3,5-xylyl)phosphino)-1,1′-binaphthyl, an R-configuration is obtainable.

The amount of the optically active phosphine ligand to be used, is usually within a range of from 0.001 to 1 mol equivalent, preferably from 0.002 to 0.1 mol equivalent, based on 1 mol equivalent of the allyloxy compound.

The above palladium compound may, for example, be palladium chloride, palladium acetate, dichlorobis(triphenylphosphine)palladium, tetrakis(triphenylphosphine)palladium, di-μ-chlorobis[(η-allyl)palladium], bis[(acetylacetonate)palladium], dichlorobis[(benzonitrile)palladium], palladium propionate, tris(dibenzylidene acetone)dipalladium or [1,1′-bis(diphenylphosphino)ferrocene]palladium chloride. Among them, dichlorobis(triphenylphosphine)palladium, di-μ-chlorobis[(η-allyl)palladium] and tris(dibenzylidene acetone)dipalladium are preferred.

The amount of the palladium compound to be used is usually within a range of from 0.1 to 3 mol equivalent, preferably from 0.9 to 1.2 mol equivalent, based on 1 mol equivalent of the optically active phosphine ligand. It is considered that such an optically active phosphine ligand forms a catalyst by coexisting with the above palladium compound.

With respect to the hydrogenated compound of the formula (3), the lower the acid dissociation constant (pKa) of hydrogen at the reaction point, in water, the higher the reaction rate, and pKa is preferably at most 16, more preferably at most 13. Such a hydrogenated compound may, for example, be an ester such as dimethyl malonate, diethyl malonate, ethyl acetoacetate, ethyl 2-fluoroacetoacetate, ethyl nitroacetate or ethyl fluoroacetate; a diketone such as acetylacetone; a nitrile such as molononitrile or ethyl cyanoacetate; a nitro compound such as nitromethane or nitroethane; an imide such as succinic imide or phthalic imide; a secondary amine such as diethylamine or dibenzylamine; or a thioacetic acid.

The amount of the hydrogenated compound to be used is usually within a range of from 0.1 to 3 mol equivalent, preferably from 0.9 to 1.2 mol equivalent, based on the allyloxy compound.

As the tertiary amine of the formula (5), it is possible to use an optional tertiary amine. In the formula (5), each of R¹¹, R¹² and R¹³ independently may be a C₂₋₁₂ aliphatic group (e.g. a hydrocarbon group such as an alkyl group, or a hydrocarbon group containing an unsaturated bond such as an allyl group) or a C₂₋₁₂ substituted aliphatic group (e.g. a substituted hydrocarbon group such as a benzyl group or a phenethyl group), which may be linear, branched or cyclic, or a C₆₋₁₀ aromatic group (such as a phenyl group or a naphthyl group) or a C₆₋₁₀ substituted aromatic group (such as a tolyl group or a xylyl group). The tertiary amine may, preferably, be a linear alkylamine such as triethylamine, tripropylamine, tributylamine, tripentylamine or trioctylamine; a branched alkylamine such as diisopropylethylamine; an aniline such as dimethyl aniline; a benzylamine such as dimethyl benzylamine, an allylamine such as triallylamine, a diamine such as tetramethyl ethylene diamine or an alicyclic amine such as 1,8-diazabicyclo[5.4.0]-7-undecene (DBU).

The amount of the tertiary amine to be used, is not particularly limited as long as it is the amount which does not interrupt the reaction and does not cause a side reaction. However, the amount is usually within a range of from 0.1 to 10 mol equivalent, preferably from 0.5 to 5 mol equivalent, more preferably from 0.9 to 1.1 mol equivalent, based on the hydrogenated compound.

The order of adding the allyloxy compound, the optically active phosphine ligand, the palladium compound, the hydrogenated compound and the tertiary amine, may be changed in any order, but it is preferred to dropwise add a mixture of the hydrogenated compound and the tertiary amine to a mixture of the optically active phosphine ligand, the palladium compound and the allyloxy compound.

The present reaction may be carried out without any solvent, but usually, it is preferred to use a solvent for the reaction.

As the solvent, water or an organic solvent is used, but it is not particularly limited as long as it is stable under the reaction conditions, and it does not interrupt the objective reaction. It is possible to use, for example, an alcohol (such as ethanol, propanol, butanol or octanol), a cellosolve (such as methoxyethanol or ethoxyethanol), an aprotic polar organic solvent (such as dimethyl formamide, dimethyl sulfoxide, dimethyl acetamide, tetramethyl urea, sulfolane, N-methyl pyrrolidone or N,N-dimethyl imidazolidinone), an ether (such as diethyl ether, diisopropyl ether, t-butyl methyl ether, tetrahydrofuran or dioxane), an aliphatic hydrocarbon (such as pentane, hexane, c-hexane, octane, decane, decalin or petroleum ether), an aromatic hydrocarbon (such as benzene, chlorobenzene, o-dichlorobenzene, nitrobenzene, toluene, xylene, mesitylene or tetralin), a halogenated hydrocarbon (such as chloroform, dichloromethane, dichloroethane or carbon tetrachloride), a ketone (such as acetone, methyl ethyl ketone, methyl butyl ketone or methyl isobutyl ketone), a low aliphatic acid ester (such as methyl acetate, ethyl acetate, butyl acetate or methyl propionate), an alkoxyalkane (such as dimethoxyethane or diethoxyethane) or a nitrile (such as acetonitrile, propionitrile or butylonitrile).

The above solvents may be used alone or in combination as a mixture of two or more of them.

Further, it is possible to use such a solvent as a nonaqueous solvent by using a proper dehydrating agent or a desiccant, as the case requires.

The optical purity of the optically active allyl compound as the product depends on the type of a solvent. The preferred solvent may, for example, be a halogenated hydrocarbon, but other than that, a preferred solvent may exist.

The amount of the reaction solvent to be used, is usually within a range of from 1 to 200 times by weight more preferably from 3 to 10 times by weight, based on the allyloxy compound.

The reaction temperature is possibly be at from −100° C. to the boiling point of the solvent to be used, but it is preferably from −50° C. to 50° C., more preferably from −10° C. to 20° C.

The reaction time varies depending on the reaction temperature and the pKa of the hydrogenated compound, and it may not simply be determined. However, in a case where the reaction temperature is 0° C. and the pKa of the hydrogenated compound is 10, it is enough to carry out the reaction for 1 hour.

After the reaction, water is added, followed by extraction with a proper solvent, and the solvent is concentrated under reduced pressure to isolate the desired optically active allyl compound. It is possible to isolate the highly purified optically active allyl compound by purification such as recrystallization, distillation or silica gel column chromatography, as the case requires.

Further, from the viewpoint of operation safety, it is preferred to carry out the reaction in an atmosphere of an inert gas such as nitrogen, argon or helium.

### EXAMPLES

Now, the present invention will be described in further detail with reference to Examples, but the present invention is by no means restricted by the following Examples.

### Examples 1 to 11

Into a glass reactor which was flushed with nitrogen, 0.47 mmol of an optically active phosphine ligand and 0.20 mmol of di-μ-chlorobis[(η-allyl)palladium] were put, and 5 g of methylene chloride was added to dissolve them. Then, 7.9 mmol of an allyloxy compound was added, followed by stirring at 0° C. for 10 minutes. On the other hand, into another glass reactor which was flushed with nitrogen, 7.9 mmol of a hydrogenated compound and 7.9 mmol of a tertiary amine were put, and 3 g of methylene chloride was added to dissolve them. The solution at that time was visually observed if it was gelled. The above two solutions were mixed at 0° C., followed by a reaction for one hour. 5 g of water was added to the reaction solution, followed by stirring, and then, the solution was subjected to liquid separation. The organic phase was concentrated under reduced pressure. The concentrated liquid was purified by silica gel column chromatography (silica gel: 30 g, developing solution: hexane/ethyl acetate=80/20), to obtain an optically active allyl compound. A part of the product was used for HPLC analysis using an optically active column, to determine the optical purity.

### Comparative Examples 1 to 4

Into a glass reactor which was flushed with nitrogen, 0.47 mmol of an optically active phosphine ligand and 0.20 mmol of di-μ-chlorobis[(η-allyl)palladium] were put, and 5 g of methylene chloride was added to dissolve them. Then, 7.9 mmol of an allyloxy compound was added, followed by stirring at 0° C. for 10 minutes. On the other hand, into another glass reactor which was flushed with nitrogen, 7.9 mmol of a hydrogenated compound was put, and 3 g of methylene chloride was added to dissolve it. 7.9 mmol of a base was added thereto. The solution at that time was visually observed if it was gelled. The above two solutions were mixed at 0° C., followed by a reaction for one hour. 5 g of water was added to the reaction solution, followed by stirring, and then, the solution was subjected to liquid separation. The organic phase was concentrated under reduced pressure. The concentrated liquid was purified by silica gel column chromatography (silica gel: 30 g, developing solution: hexane/ethyl acetate=80/20), to obtain an optically active allyl compound. A part of the product was used for HPLC analysis using an optically active column, to determine the optical purity.

### Comparative Example 5

Into a glass reactor which was flushed with nitrogen, 0.47 mmol of an optically active phosphine ligand and 0.20 mmol of di-μ-chlorobis[(η-allyl)palladium] were put, and 5 g of methylene chloride was added to dissolve them. Then, 7.9 mmol of an allyloxy compound was added, followed by stirring at 0° C. for 10 minutes. On the other hand, into another glass reactor which was flushed with nitrogen, 7.9 mmol of a hydrogenated compound was put, and 3 g of methylene chloride was added to dissolve it. 7.9 mmol of sodium hydride was added thereto. The solution at that time was visually observed if it was gelled. 7.9 mmol of tetra-n-hexylammonium bromide was added to the solution. The above two solutions were mixed at 0° C., followed by a reaction for one hour. 5 g of water was added to the reaction solution, followed by stirring, and then, the solution was subjected to liquid separation. The organic phase was concentrated under reduced pressure. The concentrated liquid was purified by silica gel column chromatography (silica gel: 30 g, developing solution: hexane/ethyl acetate=80/20), to obtain an optically active allyl compound. A part of the product was used for HPLC analysis using an optically active column, to determine the optical purity.

The results of Examples and Comparative Examples are shown in Tables 1 and 2. Further, in Tables, Et represents an ethyl group, n-Pr a n-propyl group, I-Pr an isopropyl group, c-Pr a cyclopropyl group, n-Bu a n-butyl group, s-Bu a secondary butyl group, i-Bu an isobutyl group, t-Bu a tertiary butyl group, c-Bu a cyclobutyl group, n-Pen a n-pentyl group, c-Pen a cyclopentyl group, n-Hex a n-hexyl group, c-Hex a cyclohexyl group, Hep a heptyl group, Oc an octyl group, and Ph a phenyl group. Further, structural formulae corresponding to numbers in Tables, are as follows.

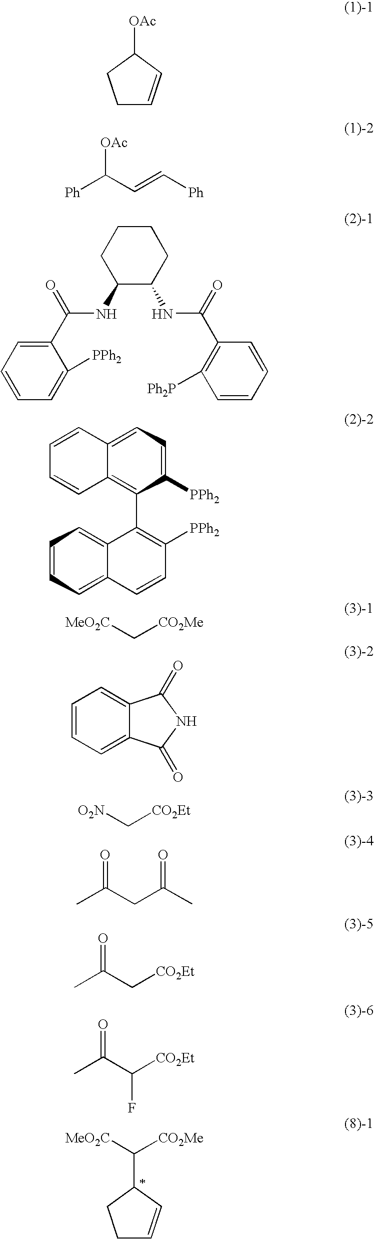

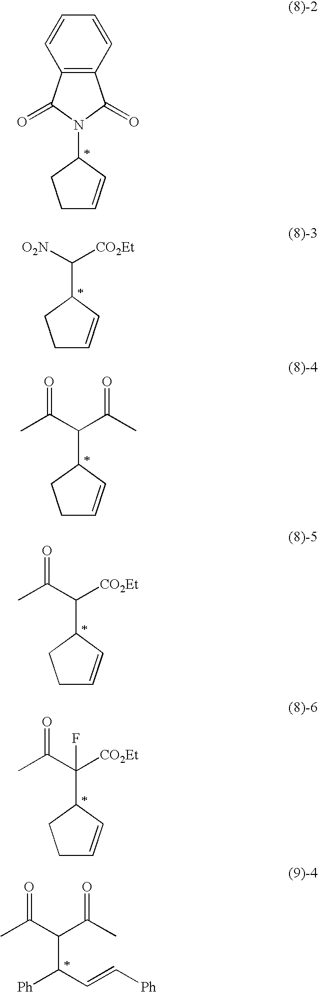

TABLE 1

| | | | | | | Optically | | | Steric |
|---|---|---|---|---|---|---|---|---|---|
| | | | Hydroge- | | | active | | Optical | configu- |
| | Allyloxy | | nated | | | allyl | Yield | purity | ration |
| Example | compound | Ligand | compound | Base | Gelation | compound | (%) | (% ee) | (R/S) |
| 1 | (1)-1 | (2)-1 | (3)-1 | n-Pr₃N | None | (8)-1 | 15 | 97 | R |
| 2 | (1)-1 | (2)-1 | (3)-2 | n-Pr₃N | None | (8)-2 | 48 | 99 | R |
| 3 | (1)-1 | (2)-1 | (3)-3 | n-Pr₃N | None | (8)-3 | 94 | 95 | R |
| 4 | (1)-1 | (2)-1 | (3)-4 | n-Pr₃N | None | (8)-4 | 88 | 100 | R |
| 5 | (1)-1 | (2)-1 | (3)-5 | n-Pr₃N | None | (8)-5 | 92 | 92 | R |
| 6 | (1)-1 | (2)-1 | (3)-6 | n-Pr₃N | None | (8)-6 | 92 | 98 | R |
| 7 | (1)-1 | (2)-1 | (3)-6 | Et₃N | None | (8)-6 | 90 | 92 | R |
| 8 | (1)-1 | (2)-1 | (3)-6 | n-Oc₃N | None | (8)-6 | 92 | 99 | R |
| 9 | (1)-1 | (2)-1 | (3)-6 | i-Pr₂EtN | None | (8)-6 | 98 | 98 | R |
| 10 | (1)-2 | (2)-1 | (3)-4 | n-Pr₃N | None | (9)-4 | 90 | 100 | R |
| 11 | (1)-2 | (2)-2 | (3)-4 | n-Pr₃N | None | (9)-4 | 90 | 90 | R |

TABLE 2

| | | | | | | Optically | | | Steric |
|---|---|---|---|---|---|---|---|---|---|
| Compara- | | | Hydroge- | | | active | | Optical | configu- |
| tive | Allyloxy | | nated | | | allyl | Yield | purity | ration |
| Example | compound | Ligand | compound | Base | Gelation | compound | (%) | (% ee) | (R/S) |
| 1 | (1)-1 | (2)-1 | (3)-6 | NaH | Observed | (8)-6 | 65 | 50 | R |
| 2 | (1)-1 | (2)-1 | (3)-5 | *1 | None | (8)-5 | 94 | 85 | R |
| 3 | (1)-2 | (2)-2 | (3)-4 | *1 | None | (9)-4 | 90 | 86 | R |
| 4 | (1)-1 | (2)-1 | (3)-4 | Cs₂CO₃ | Slurry | (8)-4 | 80 | 100 | R |
| 5 | (1)-1 | (2)-1 | (3)-6 | *2 | None | (8)-6 | 87 | 92 | R |

*1: N,O-bis(trimethylsilyl)acetamide
*2: NaH/n-Hex₄NBr

By comparing Examples 6 to 9 with Comparative Example 1, it is evident that with the process using the tertiary amine of the present invention, the reaction system does not become gelled, and the obtainable optically active ally compound has a high optical purity.

By comparing Example 5 with Comparative Example 2, it is evident that with the process using the tertiary amine of the present invention, the obtainable optically active ally compound has a high optical purity.

By comparing Example 11 with Comparative Example 3, it is evident that with the process using the tertiary amine of the present invention, the obtainable optically active ally compound has a high optical purity.

By comparing Examples 6 to 9 with Comparative Examples 5, it is evident that with the process using the tertiary amine of the present invention, the reaction system does not become gelled, and the obtainable optically active ally compound has a high optical purity.

### INDUSTRIAL APPLICABILITY

It is possible to use the present invention as a novel process for producing an optically active allyl compound which is useful as an intermediate raw material for e.g. pharmaceutical products.

The entire disclosure of Japanese Patent Application No. 2006-030964 filed on Feb. 8, 2006 including specification, claims and summary is incorporated herein by reference in its entirety.

1. A process for producing an optically active allyl compound of the formula (4): 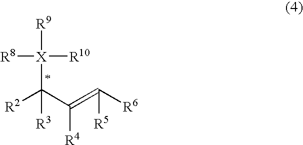
(wherein “*” represents an asymmetric carbon atom), which comprises reacting an allyloxy compound of the formula 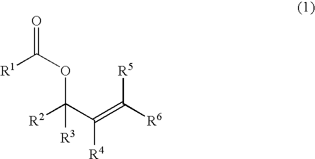
(wherein R¹ is a C₁₋₆ alkyl group or a C₁₋₆ alkoxyl group, and each of R², R³, R⁴, R⁵ and R⁶ independently is a C₁₋₆ alkyl group which may be linear, branched or cyclic, a hydrogen atom or a C₆₋₁₂ aromatic group, provided that R² and R⁶ may be located in the same ring) with a hydrogenated compound of the formula (3): 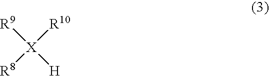
(wherein X is a carbon atom, an oxygen atom, a sulfur atom or a nitrogen atom, and each of R⁸, R⁹ and R¹⁰ independently is a C₁₋₂₄ alkyl group which may be linear, branched or cyclic, a C₁₋₂₄ alkylcarbonyl group which may be branched or cyclic, a C₁₋₂₄ alkoxycarbonyl group which may be branched or cyclic, a hydrogen atom, a halogen atom or a C₆₋₁₀ aromatic group, or two of R⁸, R⁹ and R¹⁰ may together form a ring containing one or two carbonyl groups), in the presence of a palladium compound and an optically active phosphine ligand of the formula (2): 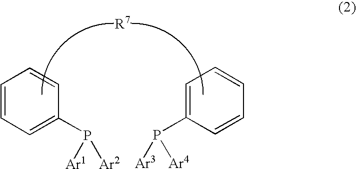
(wherein each of Ar¹, Ar², Ar³ and Ar⁴ independently is a C₆₋₁₀ aromatic group, and R⁷ is a structure having at least one asymmetric center or axial chirality), wherein a tertiary amine of the formula (5): 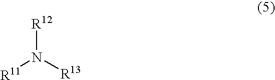
(wherein each of R¹¹, R¹² and R¹³ independently is a C₂₋₁₂ aliphatic group or a C₂₋₁₂ substituted aliphatic group, which may be linear, branched or cyclic, or a C₆₋₁₀ aromatic group or a C₆₋₁₀ substituted aromatic group) is present in the above reaction system.

2. The process according to claim 1, wherein the optically active phosphine ligand is a compound of the formula (6):

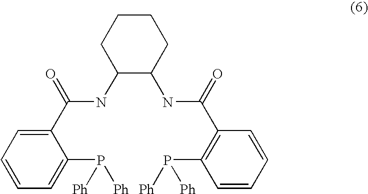

3. The process according to claim 1, wherein the optically active phosphine ligand is a compound of the formula (7):

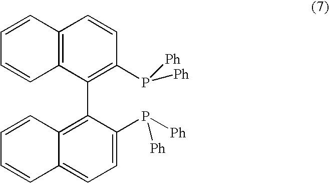

4. The process according to claim 1, wherein the allyloxy compound is cyclopentenyl acetate of the formula (8):

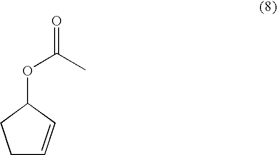

5. The process according to claim 1, wherein the allyloxy compound is a compound of the formula (9):

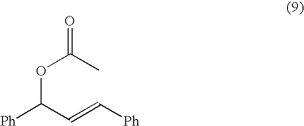

6. The process according to claim 1, wherein the tertiary amine is tri-n-propylamine.

7. The process according to claim 1, wherein the tertiary amine is tri-n-octylamine.

8. The process according to claim 1, wherein the tertiary amine is diisopropylethylamine.

9. The process according to claim 4, wherein the hydrogenated compound is a compound of the formula (10):

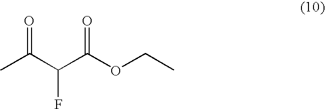

10. The process according to claim 1, wherein the hydrogenated compound has a pKa of at most 16 in water.
